(19) United States
(12) Patent Application Publication
(10) Pub. No.: US 2025/0140401 A1
(43) Pub. Date: May 1, 2025

(54) INFORMATION PROCESSING APPARATUS, METHOD FOR CONTROLLING INFORMATION PROCESSING APPARATUS, AND COMPUTER PROGRAM

(71) Applicant: ONLINEDOCTOR.COM CO., LTD., Minato-ku, Tokyo (JP)

(72) Inventor: Mikihiro SUZUKI, Minato-ku, Tokyo (JP)

(21) Appl. No.: 18/835,529

(22) PCT Filed: Oct. 21, 2022
(86) PCT No.: PCT/JP2022/039271

§ 371 Date: Aug. 2, 2024

(30) Foreign Application Priority Data
Feb. 4, 2022 (JP) 2022-016272
Mar. 30, 2022 (JP) 2022-057321

(51) Int. Cl. G16H 40/67; G06V 40/16

(52) U.S. Cl. CPC G16H 40/67; G06V 40/162

(57) ABSTRACT

This information processing apparatus comprises: an acquisition means that acquires subject's biometric information acquired by a sensor provided in a first terminal attached to the subject, and terminal information including the type of the first terminal; a storage means that stores the biometric information and the terminal information that have been acquired; a display control means that displays the biometric information and the terminal information on a display means of a second terminal different from the first terminal; an acceptance means that accepts control information with respect to the first terminal from the second terminal; and an instruction means that instructs the first terminal to control the first terminal on the basis of the control information that has been accepted.

### TECHNICAL FIELD

The present invention relates to an information processing apparatus, a method for controlling the information processing apparatus, and a computer program.

### BACKGROUND ART

Conventionally, for operating online medical examinations for patients residing in remote areas, there has been technology in which patient health information is collected on a doctor's computer via an intermediary server (see Patent Document 1, for example).

### CITATION LIST

### Patent Document

Patent Document 1: Japanese Unexamined Patent Application, Publication No. 2022-16085

### DISCLOSURE OF THE INVENTION

### Problems to be Solved by the Invention

In face-to-face medical examinations, doctors can accurately obtain biometric information of patients and the like by appropriately operating various medical devices, whereas in online medical examinations, it is difficult for doctors to accurately obtain biometric information of patients and the like because doctors and patients and the like are away from each other.

An object of the present invention, which has been made in such a background, is to provide a technique that allows medical personnel to accurately obtain biometric information of patients and the like in online medical examinations and the like.

### Means for Solving the Problems

In order to achieve the aforementioned object, an aspect of the present invention is to provide an information processing apparatus including:
an acquisition unit that acquires biometric information of a subject acquired by a sensor provided in a first terminal attached to the subject and terminal information including a type of the first terminal;
a storage unit that stores the biometric information and the terminal information that have been acquired;
a display control unit that displays the biometric information and the terminal information on a display unit of a second terminal that is different from the first terminal;
a reception unit that receives from the second terminal control information for the first terminal; and
an instruction unit that instructs the first terminal to control the first terminal based on the control information that has been received.

### Effects of the Invention

According to the present invention, it is possible for medical personnel and the like to accurately obtain biometric information of patients and the like in online medical examinations and the like.

### BRIEF DESCRIPTION OF THE DRAWINGS

FIG. 1 is a diagram showing an overview of an embodiment;

FIG. 2 is a diagram showing an overview of the configuration of a biometric information acquisition system;

FIG. 3 is a block diagram showing the hardware configuration of a server;

FIG. 4 is a functional block diagram showing an example of the functional configuration of the server;

FIG. 5 is a flowchart showing an example of biometric information acquisition processing;

FIG. 6 is a diagram showing an application example (1) in online medical examinations of internal medicine;

FIG. 7 is a diagram showing an application example (2) in online medical examinations of internal medicine;

FIG. 8 is a diagram showing an application example (3) in online medical examinations of internal medicine;

FIG. 9 is a diagram showing an application example in online ophthalmology examinations;

FIG. 10 is a diagram showing an application example in online otolaryngology examinations; and

FIG. 11 is a diagram showing an application example in online dermatology examinations.

### PREFERRED MODE FOR CARRYING OUT THE INVENTION

### Embodiment

### <Overview>

Currently, in online medical examinations, various devices, such as blood pressure monitors, are used to acquire biometric information on patients and the acquired results (e.g., blood pressure readings displayed on a blood pressure monitor) are sent to a doctor via a web conferencing system. Some of these devices automatically transmit the patient's biometric information to the doctor's terminal via a network, but specifications vary depending on the application and manufacturer. Therefore, in order for the doctor to obtain the patient's biometric information automatically, the patient has to use a device designated by the doctor. This embodiment describes an example of using protocols, APIs, and SDKs for information that can be installed on any device to acquire a patient's biometric information independently of the type of device.

Besides, the biometric information (patient's information) that can be acquired in online medical examinations is currently limited; for example, heartbeat, breathing sound, and abdominal sounds cannot be heard as planned by the doctor. This causes a risk of misdiagnosis in online medical examinations, and thus a need for face-to-face medical examinations to accurately obtain a patient's biometric information.

In contrast, the biometric information acquisition system according to this embodiment acquires the patient's biometric information in real time during online medical examination. The doctor can then select the type of biometric information to be acquired (e.g., blood pressure and heartbeat) or the sensor used to acquire the biometric information. In addition, the patient's wearable device is instructed to acquire the selected biometric information. Such interactive data communication allows the same level of (accurate) biometric information as in face-to-face medical examinations to be acquired in online medical examination. This embodiment will be described below with reference to the accompanying drawings.

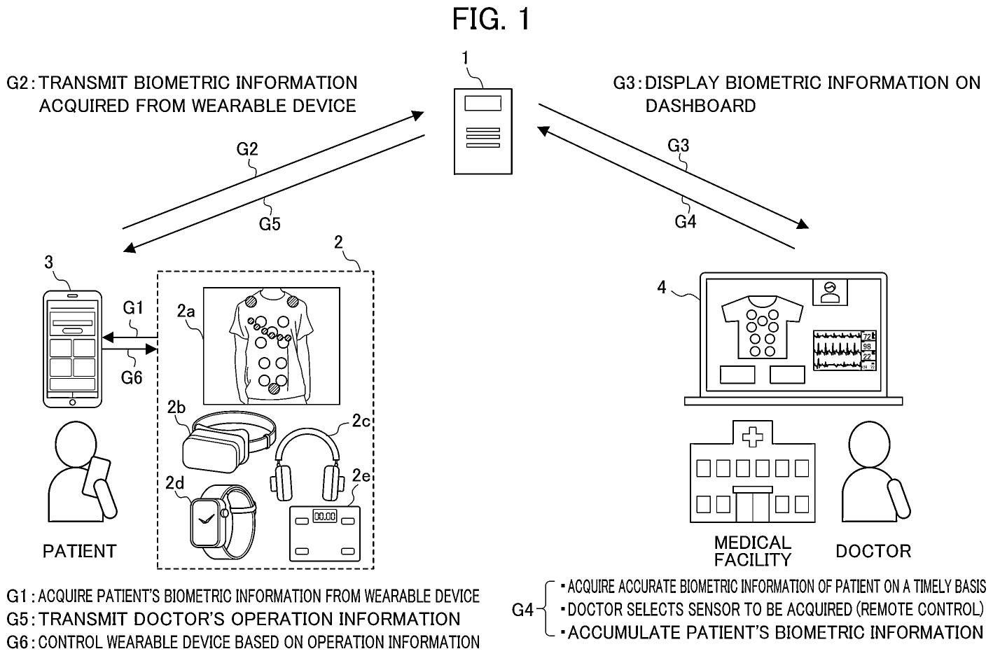

FIG. 1 is a diagram showing an overview of a biometric information acquisition system to which a server 1 (information processing apparatus) according to this embodiment is applied. In this example, when a patient and a doctor (medical facility) conduct online medical examination using a given web conferencing system, the patient's biometric information is acquired according to the doctor's intention. FIG. 1 shows an example of various data communication at the server 1, wearable device 2, patient terminal 3, and medical facility terminal 4 of the biometric information acquisition system of this embodiment.

As the reference numeral G1 shows, the patient's biometric information is acquired by a predetermined sensor provided in the wearable device 2 worn by the patient. The acquired biometric information is transmitted to the patient terminal 3 by near field communication. The communication method for the near field communication is not limited, but may be, for example, Bluetooth (registered trademark), Wi-Fi (registered trademark), or other communication methods. As the reference numeral G2 shows, the patient terminal 3 transmits biometric information acquired by the wearable device 2 to the server 1. As the reference numeral G3 shows, the server 1 displays the biometric information on the display (display unit) of the medical facility terminal 4 (dashboard) operated by the doctor. This allows the doctor to acquire accurate biometric information of a patient on a timely basis by referring to the patient's biometric information displayed on the medical facility terminal 4. The doctor can not only get the patient's biometric information but also select which of the sensors in the wearable device 2 will be used to acquire biometric information. As the reference numeral G4 shows, upon reception of an instruction to select a sensor, the medical facility terminal 4 transmits the operation information related to the instruction to the server 1. The patient's biometric information is not only displayed on the medical facility terminal 4, but also stored in the storage unit of the server 1. As the reference numeral G5 shows, upon reception of operation information from the medical facility terminal 4, the server 1 transmits the operation information to the patient terminal 3. As the reference numeral G6 shows, the patient terminal 3 controls the wearable device 2 according to the operation information described above.

Biometric information is information about a living body (patient) that is used in medical examination and tests, and includes sounds produced by the living body (e.g., abdomen, embryo, and heart), pulse wave, body temperature, heartbeat, electrocardiogram, blood pressure, respiratory rate, hearing, electroencephalogram, biological surface (e.g., skin) or internal (e.g., intraoral) information (e.g., visual information), fundus oculi, cornea, intraocular pressure, eyesight, weight, height, muscle mass, BMI, basic metabolism, blood glucose level, and blood oxygen level (concentration). The biometric information also includes the results of various analyses of the aforementioned data. To obtain the biometric information described above, the wearable device 2 (device) includes at least one or more selected from the following different sensors: sound sensors, temperature sensors, pressure sensors, optical sensors, ultrasonic sensors, electroencephalogram sensors, pulse wave sensors, electrocardiographic sensors, pressure sensors, and imaging sensors.

Examples of the wearable device 2 include devices 2a to 2e. The devices 2a to 2e each have a module that transmits biometric information acquired by each device. The devices 2a to 2e are intended to be purchased by patients for online medical examination or rented or leased from a given institution.

The device 2a is a clothing-type device with various sensors. It may be a device (including sensors) that can be attached (e.g., pasted) to regular clothing. The device 2a is intended to be used mainly in online medical examination of internal medicine, and is, for example, a device that functions as a stethoscope, blood pressure monitor, electrocardiogram measurement device, and the like used in face-to-face medical examinations. The device 2a uses different sensors to acquire, for example, at least one selected from the following types of biometric information: lung, abdominal, and heart sounds, body temperature, blood pressure, electrocardiogram, heart rate, and respiratory rate. The device 2a is not limited to the short-sleeved T-shirt type shown in FIG. 1, but may be a long-sleeved shirt type, and may also be in various forms, such as head, lower body, or full body type.

The device 2b is a goggle-type device. The device 2b is intended to be used primarily in online ophthalmology examinations and serves as, for example, an ophthalmic examination device (e.g., AutoRefkeratometer) in face-to-face medical examinations. The device 2b uses different sensors to acquire, for example, at least one selected from the following types of biometric information: fundus oculi, cornea, and intraocular pressure. The device 2b includes a light source and can also control light's ON/OFF. The device 2b may also include a display unit and can control the images displayed on the display unit.

The device 2c is a headphone-type device. The device 2c is mainly intended to be used in online otolaryngology examinations, and serves as, for example, an otoscope in face-to-face medical examinations. The device 2c uses different sensors to acquire at least one selected from the biometric information indicating the status of the tympanic membrane, outer ear, tympanometry, and hearing. The device 2c is not limited to the headphone type, and may be capable of acquiring biometric information not only about the ear but also about the nasal cavity, oral cavity, pharynx and the like located in the nose and oral cavity. The device 2c includes a light source and can also control light's ON/OFF. A disposable cover, which allows for acquisition of more detailed status of the tympanic membrane and the like, is attachable to the device 2c.

The device 2d is a wristwatch-type device. The device 2d is mainly intended to be used in online medical examinations of internal medicine and serves as, for example, a blood pressure monitor, pulse oximeter, blood glucose meter, or the like in face-to-face medical examinations. The device 2d uses different sensors to acquire at least one selected from the following biometric information: blood pressure, blood glucose level, heart rate, blood oxygen level (concentration), pulse, body load, number of steps, number of falls, location information, calorie consumption information, sleep information, voice information, breathing sound, and heartbeat sound.

The device 2e is a body composition analyzer. The device 2e is mainly intended to be used in online medical examinations of internal medicine and serves as, for example, a body composition analyzer in face-to-face medical examinations. The device 2e uses different sensors to acquire at least one selected from the following biometric information: blood glucose level, skeletal muscle percentage, bone mass, BMI, body fat percentage, body weight (ideal weight and fat-free weight), water content, basal metabolic rate, protein content, skeletal muscle percentage, muscle weight, fat weight, visceral fat grade, subcutaneous fat grade, body age, fat burning heart rate, nutrition level, and body shape. The device 2e may be combined with a wristwatch-type device shown in the device 2d (by transmitting data to the device 2d), for example, to acquire and store biometric information. Thus, the combination of the devices 2d and 2e is considered a wearable device.

### <System Configuration>

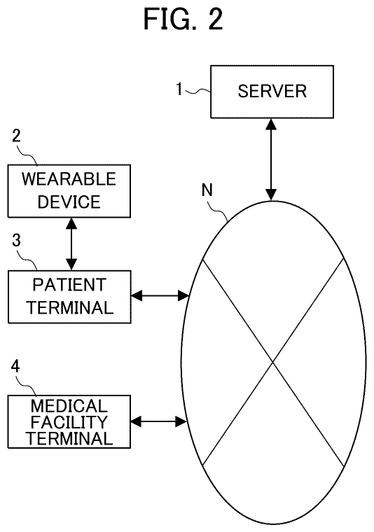

FIG. 2 is a diagram showing an overview of the system configuration of the biometric information acquisition system according to this embodiment. The biometric information acquisition system according to this embodiment includes a server 1, which performs processing, a patient terminal 3, which is operated by the patient, and a medical facility terminal 4, which displays biometric information, all interconnected via a predetermined network N such as the Internet. The wearable device 2, which acquires biometric information, and the patient terminal 3 are mutually connected by a predetermined near field communication.

The server 1 executes different types of processing in cooperation with the operations of the wearable device 2, patient terminal 3, and medical facility terminal 4. The wearable device 2 may transmit the acquired biometric information to the server 1 without through the patient terminal 3.

### <Hardware Configuration>

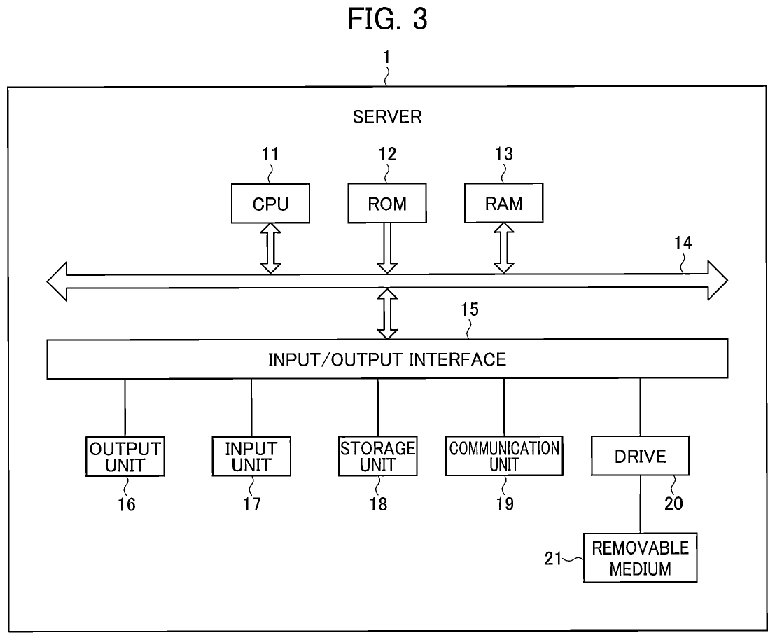

FIG. 3 is a block diagram showing the hardware configuration of the server 1 according to this embodiment. The server 1 includes a central processing unit (CPU) 11, a read only memory (ROM) 12, a random-access memory (RAM) 13, a bus 14, an input/output interface 15, an output unit 16, an input unit 17, a storage unit 18, a communication unit 19, and a drive 20.

The CPU 11 executes different types of processing according to the programs recorded in the ROM 12 or loaded into the RAM 13 from the storage unit 18. The RAM 13 also stores data and other information necessary for the CPU 11 to perform different types of processing as appropriate. The CPU 11, ROM 12, and RAM 13 are mutually connected via the bus 14. The input/output interface 15 is also connected to this bus 14.

The output unit 16, input unit 17, storage unit 18, communication unit 19, and drive 20 are connected to the input/output interface 15. The output unit 16 consists of a display, speakers, and the like, and outputs different types of information as images and sound. The input unit 17 includes a keyboard and mouse to input different types of information. The storage unit 18 includes a hard disk and a dynamic random access memory (DRAM), and stores different types of data. The communication unit 19 communicates with other devices via the network N including the Internet.

A removable medium 21 consisting of a magnetic disk, optical disk, magneto-optical disk, semiconductor memory, or the like is mounted to the drive 20 as appropriate. Programs read from the removable medium 21 by the drive 20 are installed in the storage unit 18 as needed. The removable medium 21 can also store different types of data stored in the storage unit 18 similarly to the storage unit 18.

Although not shown in the drawing, the wearable device 2, patient terminal 3, and medical facility terminal 4 have the hardware configuration shown in FIG. 3. Although not shown in the drawing, the medical facility terminal 4 has the hardware configuration shown in FIG. 3 and includes a display monitor (dashboard) that displays the patient's biometric information. The wearable device 2 also includes a transmission unit that transmits the acquired biometric information to the server 1 or patient terminal 3, a receiving unit that receives control information from the medical facility terminal 4, and a control unit that controls different sensors based on the control information.

### <Functional Configuration>

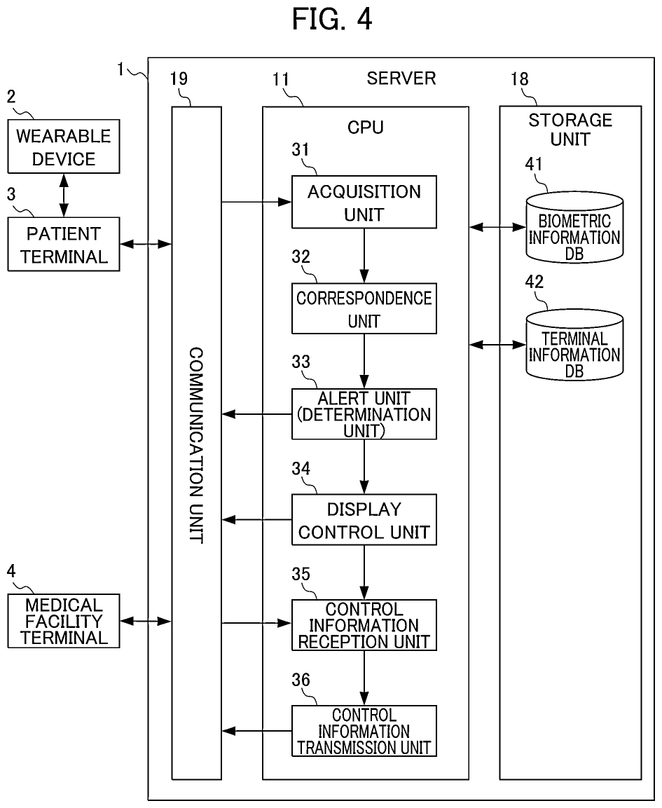

FIG. 4 is a functional block diagram showing an example of a functional configuration in the server 1 according to this embodiment.

In the CPU 11 of the server 1, during operation, the acquisition unit 31, correspondence unit 32, alert unit 33, display control unit 34, control information reception unit 35, and control information transmission unit 36 function.

The acquisition unit 31 acquires the patient's biometric information acquired by the sensors provided in the wearable device 2 (first terminal) worn by the patient (subject) and terminal information including the type of the wearable device 2. As described above, biometric information is information about a living body (patient) that is used in medical examinations and tests. The terminal information indicates the type of the wearable device 2 (e.g., which of the aforementioned devices 2a to 2e it is, or information indicating the manufacturer, product number, and the like) and at least one or more selected from the number, type, location, and the like of the sensors in the wearable device 2. Information indicating whether or not it includes a camera, lighting, and the like may also be included in the terminal information. The acquired biometric information is stored in biometric information DB41. The acquired terminal information is stored in the terminal information DB42. Although this embodiment describes an example in which multiple biometric sensors are provided in the wearable device 2, the number of sensors provided in the wearable device 2 is not limited and may be even one. Further, although this embodiment describes an example of acquiring biometric information using the wearable device 2 worn by the patient, this is not necessarily the case; for example, a device with a sensor may be attached to the patient's clothing to acquire the patient's biometric information.

The correspondence unit 32 establishes correspondences between the aforementioned biometric information and the location of the sensor that has acquired the biometric information in the aforementioned wearable device 2. This allows the display control unit 34, described below, to display on the medical facility terminal 4 an image showing the wearable device 2 and an image showing the location of the sensor that has acquired the biometric information in the wearable device 2.

The alert unit 33 presents an alert to the medical facility terminal 4 (the second terminal). For example, the alert unit 33 presents an alert when the biometric information is outside a predetermined range. The predetermined range is, for example, the range considered normal for blood pressure (it may differ depending on age and other factors). The predetermined range is stored in the aforementioned storage unit 18. The alert unit 33 may also use a classifier generated by machine learning to determine whether or not the range is within the predetermined range. For example, in the case of a lung sound, if it is a normal breathing sound (if no noise is generated) may be determined, and if it is not normal (if it is not within the predetermined range), an alert may be presented. For example, the alert unit 33 may also present alerts based on past biometric information of the patient. Since the normal value of biometric information is expected to vary from patient to patient, for example, the alert unit 33 may record the biometric information of the same patient in the storage unit 18 and present an alert when the biometric information differs by more than a predetermined value from the average (or statistical value such as median or mode) of the past biometric information. The method of presenting alerts is not limited. For example, the alert unit 33 may, for example, display the content of the alert and output an alert sound to the output unit of the medical facility terminal 4. For example, the alert unit 33 may display the alert contents, display advice based on the alert contents, output an alert sound and the like to the output unit of the patient terminal 3. Further, for example, the alert unit 33 may output alerts not only to the medical facility terminal but to the terminal of the administrator of the biometric information acquisition system according to this embodiment or to the terminals of partner institutions other than the medical facility. When an error (alert) is received, the administrator or other person, for example, may take countermeasures based on the error. For example, when the wearable device 2 is worn by a bedridden elderly person or a user of a nursing home, and a predetermined error occurs, the aforementioned administrator (person in charge of service) or a person in charge of the partner institution (e.g., a caregiver or a nurse) may perform escalation based on the error content received (e.g., contact an undertaker or guardians, or emergency doctors).

The display control unit 34 controls biometric information and terminal information so that they are displayed on the display (display unit) of the medical facility terminal 4. In this embodiment, the display control unit 34 displays an image showing the wearable device 2, an image (item) showing the location of the sensor of the wearable device 2, and biometric information acquired by the sensor on the display described above, based on the biometric information and terminal information. The image showing the wearable device 2 is, for example, in the case of a clothing-type wearable device 2, an item showing the shape of the clothing. Based on the terminal information, the display control unit 34 controls the display unit to display the operation unit (e.g., touch buttons) corresponding to operations that can be controlled by the medical facility terminal 4 (e.g., turning on/off the light). The display control unit 34 may display the biometric information and terminal information not only on the doctor's side but also on the patient terminal 3 which is on the patient's side. This allows the doctor and patient to look at the same data (e.g., biometric information) and talk to each other, thus allowing the patient to gain a deeper understanding of his/her own symptoms. There may be more than one patient terminal 3 on the patient's side and more than one medical facility terminal 4 on the doctor's side. This allows, for example, a patient to view data on another PC or mobile terminal while receiving an online medical examination on a tablet PC.

The control information reception unit 35 receives control information for the wearable device 2 from the medical facility terminal 4. For example, the control information reception unit 35 receives from the medical facility terminal 4 the designation of one or more sensors selected from the multiple sensors in the wearable device 2. For example, the control information reception unit 35 receives information on different types of control in the wearable device 2, from the medical facility terminal 4. Information on different types of control includes, for example, the on/off of the light, the image capture position or zoom-in/zoom-out, and selection of the type of biometric information to be acquired. There may be more than one medical facility terminal 4. For example, when an internist is consulting a patient in cooperation with a specialist, the specialist may perform operations (e.g., designate sensors) and the internist may view biometric and other information. It is also conceivable that a medical resident may view the situation in which the specialist is operating. Specific operating procedures and timing can be recorded and procedure can be documented, so that the same procedures can be operated the next time or later based on information registered as a record of optimal procedures or case studies, or procedures may be constructed through machine learning to more accurately understand the patient's situation.

The control information transmission unit 36 instructs (transmits instruction information to) the wearable device 2 to control the wearable device 2 based on the control information received by the control information reception unit 35 described above. For example, the control information transmission unit 36 instructs the wearable device 2 to acquire biometric information corresponding to the designated sensor. The wearable device 2 that has received control information controls different sensors and the like based on the control information. The control information of the wearable device 2 may be transmitted to the wearable device 2 via the patient terminal 3.

In the storage unit 18 of the server 1 shown in FIG. 4, biometric information DB41 and terminal information DB42 are provided. The biometric information DB41 and terminal information DB42 may store information acquired not only in-house but also, for example, by API connection to the specific external DB of an inspection device. For example, if MRI or x-ray data is to be viewed with dashboard function according to this embodiment, it may read information from a database that is not in the in-house own server.

The biometric information DB41 is a database in which biometric information acquired by the acquisition unit 31 is stored. The stored biometric information is managed, for example, by associating each patient (identification ID) with the type of sensor, biometric information values (e.g., blood pressure), date and time of acquisition, and the like. These pieces of information may be stored in an encrypted form. In this case, it is compounded based on the specific decryption key information.

The terminal information DB42 is a database in which terminal information acquired by the acquisition unit 31 is stored. The stored terminal information is managed, for example, by associating each terminal (wearable device 2) with a product number, sensor type and location, and installed functions (e.g., lighting and cameras). These pieces of information may be stored in an encrypted form. In this case, it is compounded based on the specific decryption key information.

### <Processing Details>

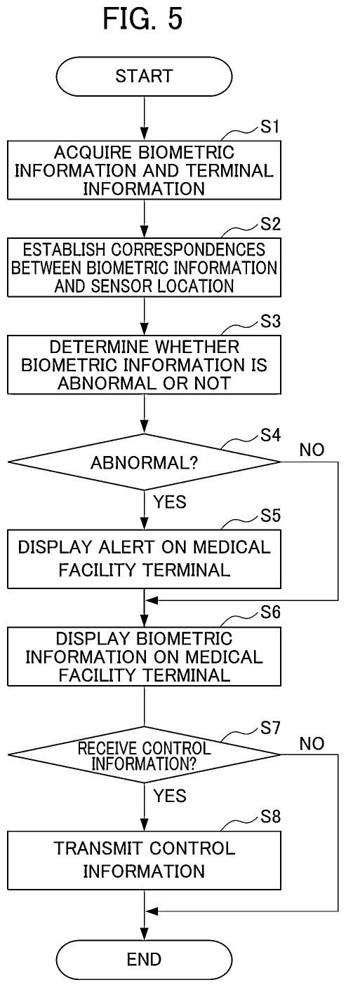

FIG. 5 shows an example of the biometric information acquisition processing according to this embodiment. Although FIG. 5 shows the processing in the server 1, the wearable device 2, patient terminal 3, medical facility terminal 4, and the like operate in cooperation with the processing in the server 1 as appropriate.

In Step S1, the acquisition unit 31 acquires the patient's biometric information acquired by a sensor provided in the wearable device 2 (first terminal) attached to the patient (subject) and the terminal information including the type of the wearable device 2.

In Step S2, the correspondence unit 32 establishes correspondences between the aforementioned biometric information and the location of the sensor that has acquired the biometric information in the aforementioned wearable device 2 (a location corresponding to a body part of the patient).

In Step S3, the alert unit 33 determines whether the biometric information described above is normal or not. For example, the alert unit 33 makes a judgment “normal” when the biometric information is within the predetermined range and a judgment “abnormal (not normal)” when the biometric information is outside the predetermined range. For example, based on the past biometric information of the patient, the alert unit 33 makes a judgment “normal” when there is a difference of less than a predetermined value from the average value of the past biometric information, and makes a judgment “abnormal” when there is a difference of greater than a predetermined value from the average value of the past biometric information.

In Step S4, the alert unit 33 determines whether the judgment in Step S3 is “abnormal” or not. If it is “abnormal,” the process proceeds to Step S5; otherwise (if it is “normal”), proceeds to Step S6.

In Step S5, the alert unit 33 presents an alert to the medical facility terminal 4. The alert unit 33 may display the aforementioned alert on both the medical facility terminal 4 and the patient terminal 3, and if necessary, on the display units of multiple medical facility terminals 4 and multiple patient terminals 3. The contents of the alert are recorded in the biometric information DB.

In Step S6, the display control unit 34 displays the biometric information and terminal information on the display of the medical facility terminal 4.

In Step S7, the control information reception unit 35 receives control information for the wearable device 2 from the medical facility terminal 4.

In Step S8, the control information transmission unit 36 transmits instruction information to the wearable device 2 to control the wearable device 2 based on the control information that has been received by the control information reception unit 35 described above. Upon reception of the instruction information, the wearable device 2 acquires the patient's biometric information according to the instruction information and transmits it to the server 1. The subsequent processing is similar to Steps S1 to S8 described above.

### <Application Examples>

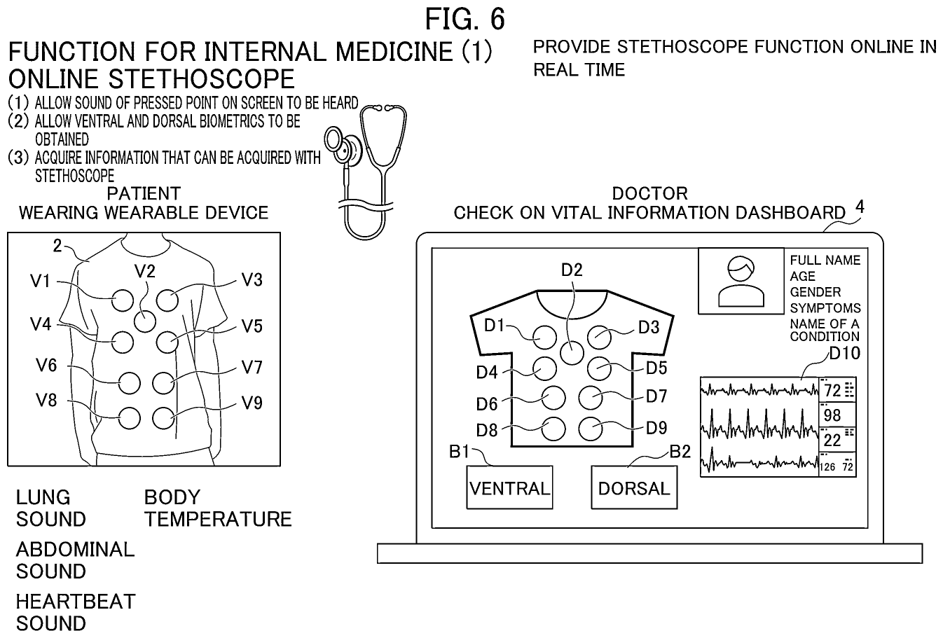

FIGS. 6 to 11 are diagrams showing the application examples of the biometric information acquisition system according to this embodiment. The doctor can check the biometric information acquired by the wearable device 2 shown in FIGS. 6 to 11 through the medical facility terminal 4 (vital information dashboard). In addition, designating different sensors, the doctor can check the biometric information (e.g., numerical information and sound information) acquired by the designated sensors.

### <<Function for Internal Medicine 1>>

FIG. 6 shows an application example (1) in online medical examination of internal medicine. As shown in FIG. 6, the patient wears a clothing-type wearable device 2 having different sensors, which allows the function of a stethoscope in face-to-face medical examinations to be provided in real time even in online medical examinations. In other words, use of the wearable device 2 shown in FIG. 6 provides the following effects.
The sound of the selected location can be heard on the screen of the medical facility terminal 4.
Biometric information on the patient's ventral and dorsal can be obtained.
Information that can be acquired with a stethoscope in face-to-face medical examinations can also be acquired in online medical examinations.

Examples of biometric information acquired by the wearable device 2 shown in FIG. 6 include lung sounds, abdominal sounds, back sounds, heart sounds, body temperature, electrocardiograms, and the like. In the example in FIG. 6, a sound sensor, temperature sensor, electrocardiographic sensor, and the like are provided at the positions indicated by the reference numerals V1 to V9. In addition, the medical facility terminal 4 displays the items indicated by the reference numerals D1 to D9 at the positions corresponding to the reference numerals V1 to V9. By selecting the desired sensor (e.g., by touching the display unit equipped with a touch panel), the doctor can grasp (confirm) the biometric information acquired by the corresponding sensor. By selecting (e.g., touching) the ventral button B1 or dorsal button B2 displayed on the display unit of the medical facility terminal 4, the user can switch between the ventral side and dorsal side of the clothing-type wearable device 2. (Different sensors are provided on the dorsal side as well as the ventral side of the wearable device 2.) The doctor can check the patient's biometric information in the area indicated by the reference numeral D10. The type and number of sensors provided in the wearable device 2 is not limited, and multiple sensors of different types may be provided.

In the example shown in FIG. 6, the following specifications are used, taking into account what is necessary for the doctor to make an accurate diagnosis.
The chest is divided into right and left.
Sensors are provided to listen to sounds in the upper, middle, and lower lung fields (12 locations in front, back, left, and right).
For the heart, sensors are provided to listen to the basal (right and left), apex, and central (sounds).

The doctor can listen to the sound of the selected part by clicking the part he/she wants to hear on the medical facility terminal 4 (e.g., tablet or PC). Suppose that the images of the wearable device 2 and sensor displayed on the medical facility terminal 4 coincide with the actual locations of the wearable device 2 and sensor. Note that actual stethoscopes come in bell and membrane types, with some listening mainly to low tones and some listening mainly to high tones; therefore, there may be a function to switch between the two as appropriate. An automatic mode may also be set up to acquire sounds in a pre-set order. If the aforementioned alert unit 33 determines that there is an abnormality, it may suggest, “Would you like to listen again to the sound at the point where you suspect there is an abnormality?” If past biometric information (e.g., breathing sounds) of the target patient exists, the previous sound and the current sound may be alternately output in order to compare them with the past (e.g., previous) sound. Hence, whether or not there is improvement can be indicated. The doctor may be able to choose whether or not to allow recording and documentation. For the wearable device 2, elastic fabric may be used so that the position of the sensor can be adjusted to some extent, even if it is worn by an overweight person. The locations of the patient's heart and lungs may be specified based on the biometric information acquired by multiple sensors.

### <<Function for Internal Medicine 2>>

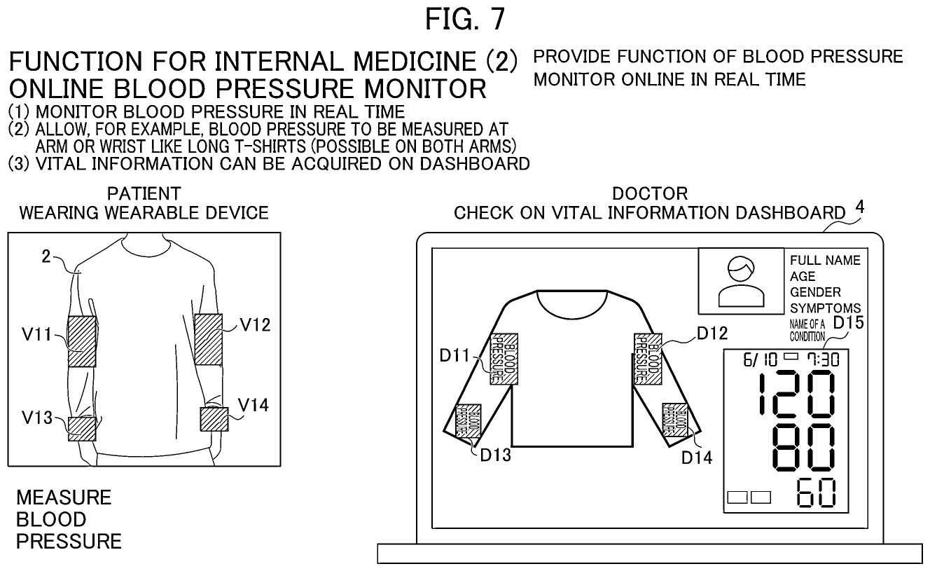

FIG. 7 is a diagram showing application example (2) in the online medical examination of internal medicine. As shown in FIG. 7, the patient wears a clothing-type wearable device 2 having different sensors, which allows the function of a blood pressure monitor in face-to-face medical examinations to be provided in real time even in online medical examinations. In other words, use of the wearable device 2 shown in FIG. 7 provides the following effects.
A patient's blood pressure and heart rate can be obtained in real time.
Blood pressure can be measured at the desired position, such as the arm or wrist.
Biometric information (blood pressure) can be checked on the medical facility terminal 4.

Examples of biometric information acquired by the wearable device 2 shown in FIG. 7 include blood pressures at different positions. In the example shown in FIG. 7, pressure sensors for measuring blood pressure are provided at the positions denoted by the reference numerals V11 to V14. The medical facility terminal 4 also displays the items denoted by the reference numerals D11 to D14 at the positions corresponding to the reference numerals V11 to V14.

The position of each blood pressure sensor may be remotely operated (controlled) by the doctor. In addition, a function may be provided that allows the doctor to remotely change the electromagnetic field if the electrodes of the sensor are weak. There may also be a function that allows the doctor to remotely adjust the sensitivity. This is because the sensor may not respond with normal sensitivity for overweight people, for example. Since blood pressure may differ between left and right, it is preferable to provide blood pressure sensors on both sides. An alert may be presented when the left-right difference in blood pressure exceeds the specified value.

### <<Function for Internal Medicine 3>>

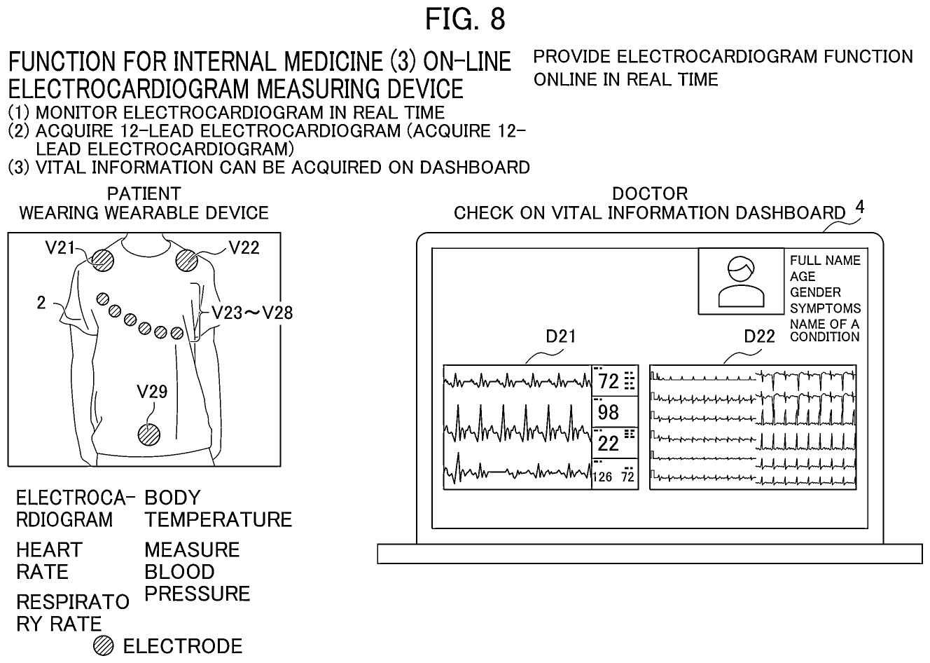

FIG. 8 is a diagram showing an application example (3) in online medical examinations of internal medicine. As shown in FIG. 8, the patient wears a clothing-type wearable device 2 having different sensors, which allows the function of an electrocardiogram measurement device in face-to-face medical examinations to be provided in real time even in online medical examinations. In other words, use of the wearable device 2 shown in FIG. 8 provides the following effects.
Electrocardiogram can be obtained in real time.
For example, an electrocardiogram in a 12-lead electrocardiogram test can be acquired (even at three points: right shoulder, left shoulder, and abdomen).
Biometric information (electrocardiogram) can be checked on the medical facility terminal 4.

Examples of biometric information acquired by the wearable device 2 shown in FIG. 8 include an electrocardiogram. In the example shown in FIG. 8, an electrocardiographic sensor is provided at the positions indicated by the reference numerals V21 to V29 (or a part of them). The medical facility terminal 4 also displays items representing the electrocardiograms indicated by the reference numerals D21 to D22. In the example shown in FIG. 8, nine sensors are shown as an example; however, for example, 20 sensors may be provided and the doctor may decide which sensor to use. This allows the use of a sensor that is installed at a position corresponding to the target part of the body (body part of the patient) for the doctor, regardless of the body shape or the like of the patient wearing the wearable device 2. The medical facility terminal 4 may display items for designation by the doctor at the positions corresponding to the reference numerals V21 to V29.

Similar to the above, a function may be provided that allows the doctor to remotely change the electromagnetic field if the electrodes of the sensor are weak. Similarly to the above, a function may be provided with which the doctor adjusts the sensitivity remotely. Note that a function may be provided that allows the doctor to remotely change the electromagnetic field in case the electrodes of the sensor are weak. Guidance on how to put on the wearable device 2 (e.g., “Please put the left side more closely ”) may also be provided on the application on the patient terminal 3. In this case, an alert may be presented to the patient side to show if the device is being worn correctly. In the event of device failure (e.g., voltage drop), the system may automatically detect such failure and alert the user. The patient's own clothing may be used as the wearable device 2 by affixing a pad with an electrocardiographic sensor to the patient's own clothing. In this case, biometric information is supposed to be transmitted from the pad to the patient terminal 3.

### <<Function for Ophthalmology>>

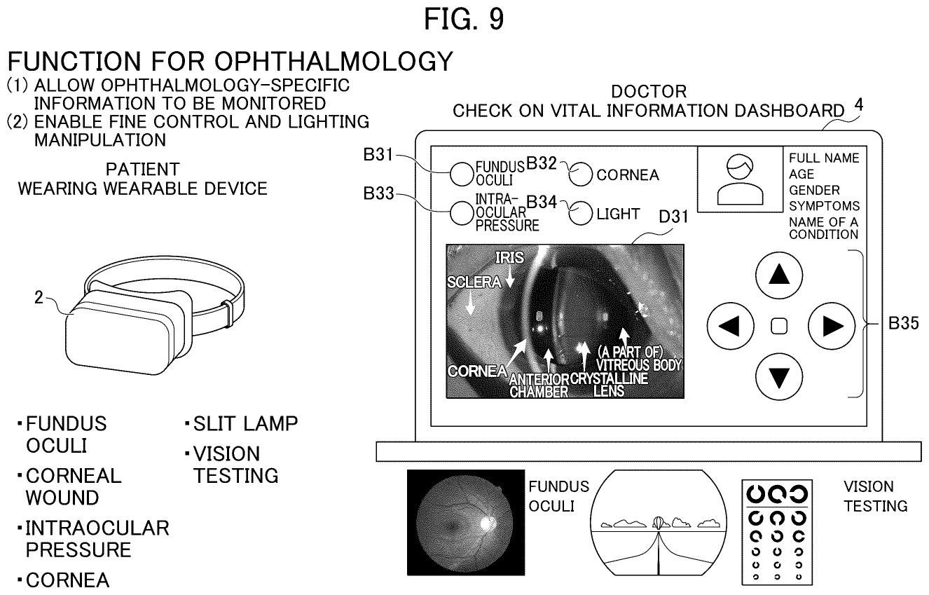

FIG. 9 shows an application example in online ophthalmology examinations. As shown in FIG. 9, the patient wears a goggle-type wearable device 2 having different sensors, which allows the function of an ophthalmic examination device in face-to-face medical examinations to be provided in real time even in online medical examinations. In other words, use of the wearable device 2 shown in FIG. 9 provides the following effects.
Information specific to ophthalmology can be obtained in real time.
Detailed operations and lighting manipulation can be performed.

Examples of biometric information acquired by the wearable device 2 shown in FIG. 9 include information about the fundus oculi, slit lamp, corneal wound, eyesight, intraocular pressure, and corneal condition. In the example shown in FIG. 9, the medical facility terminal 4 displays images related to the eye indicated by the reference numeral D31 and the intraocular pressure (not shown) described above. Examples of images related to the eye include the sclera, iris, cornea, anterior chamber, crystalline lens, and vitreous body (or a part of them). The type of image indicated by the reference numeral D31 can be changed when the physician selects the buttons indicated by the reference numerals B31 to B33. The on/off of the light (lighting) in the wearable device 2 can be controlled when the doctor presses (touches) the button indicated by the reference numeral B34. In addition, the ranges or positions included in the image indicated by the reference numeral D31 can be changed when the doctor presses (or touches) the button indicated by the reference numeral B35. Note that for example, the doctor's operation can control the display of an image for vision testing, etc. on the display unit of the wearable device 2.

Note that the position of the patient's eyelashes, for example, may be recognized to check whether the patient is wearing the wearable device 2 correctly, and output an alert if the patient is not wearing it correctly.

### <<Function for Internal Medicine and Otolaryngology>>

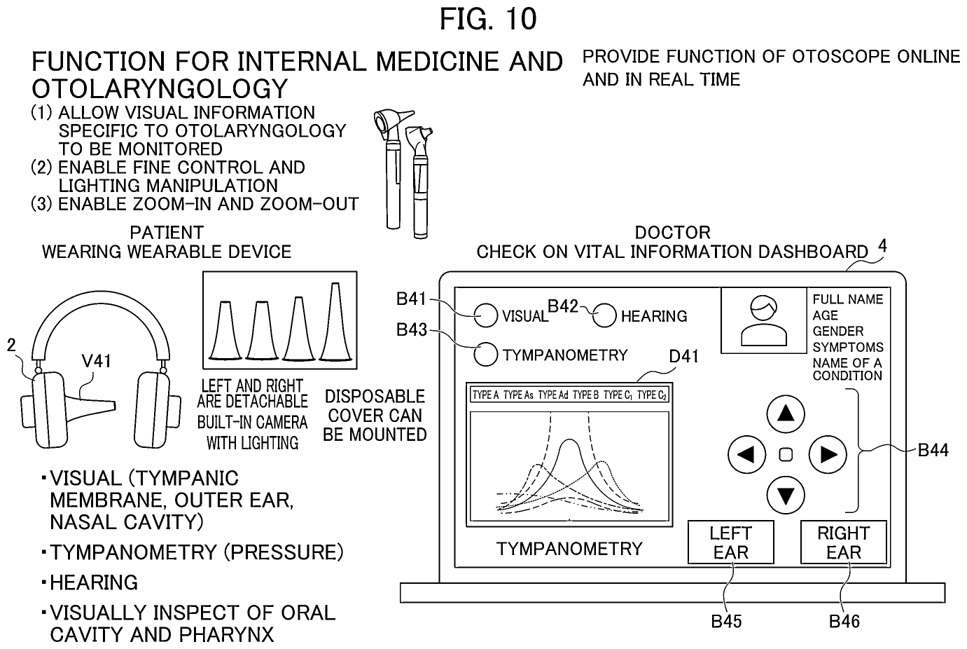

FIG. 10 shows an application example in online otolaryngology examinations. As shown in FIG. 10, the patient wears a headphone-type wearable device 2 having different sensors, which allows the function of otoscopy in face-to-face medical examinations to be provided in real time even in online medical examinations. In other words, use of the wearable device 2 shown in FIG. 10 provides the following effects.
Visual information specific to otolaryngology can be obtained in real time.
Detailed operations and lighting manipulation can be performed.
Zoom-in and zoom-out can be performed.

Examples of biometric information acquired by the wearable device 2 shown in FIG. 10 include information about the visual image (e.g., tympanic membrane, external ear, and nasal cavity), tympanometry (pressure), hearing, oral cavity, and larynx condition. In the example shown in FIG. 10, the disposable cover indicated by the reference numeral V41 can be mounted. The disposable cover is supposed to include, for example, a camera (image-capturing unit) and lighting. The medical facility terminal 4 also displays an image representing tympanometry indicated by the reference numeral D41. The type of image (e.g., visual image, hearing, and tympanometry) indicated by the reference numeral D41 can be changed when the doctor selects the buttons indicated by the reference numerals B41 to B43. When the visual image is displayed, a button may be displayed to control the on/off of the light (lighting) in the wearable device 2. For hearing, for example, a button to control the wearable device 2 so that it emits a predetermined sound to test hearing may be displayed. In addition, the ranges or positions included in the image indicated by the reference numeral D41 can be changed when the doctor presses (or touches) the button indicated by the reference numeral B44.

Note that whether the biometric information is about either the left or right ear may be recognized and displayed. In this case, at the time of wearing the wearable device 2, the patient is supposed to wear the wearable device 2, distinguishing its left and right sides. An automatic mode may also be set up to acquire sounds in a pre-set order. An alert may also be presented if the ambient sound is loud (e.g., above a predetermined decibel). For example, if the TV is too loud, it is expected to alert the user to “turn it off”, for example.

### <<Function for Dermatology>>

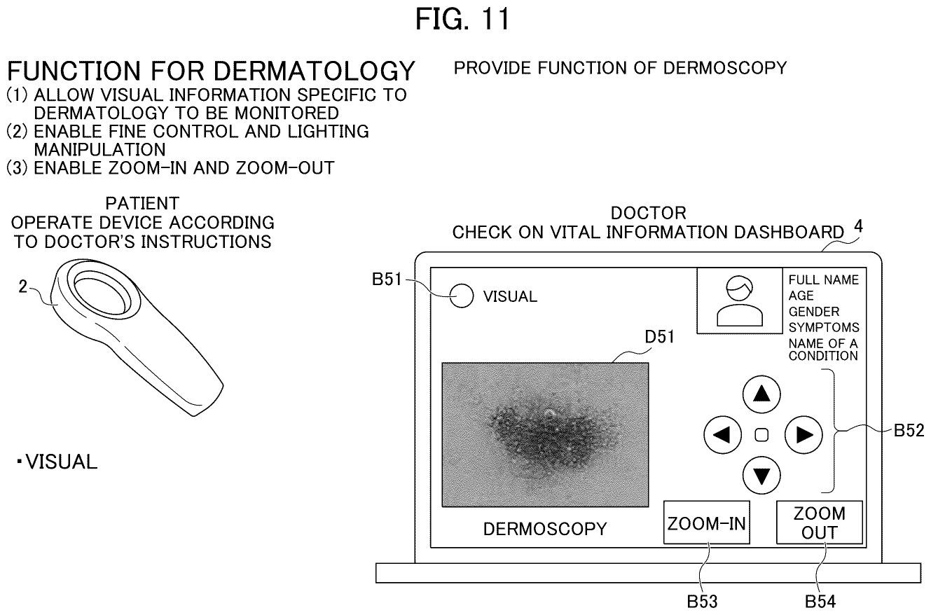

FIG. 11 shows an application example in online dermatology examinations. As shown in FIG. 11, the patient wears (operates) a wearable device 2 having different sensors, which allows the function of dermoscopy in face-to-face medical examinations to be provided in real time even in online medical examinations. The wearable device 2 shown in FIG. 11 is supposed to include, for example, a camera (image-capturing unit) and lighting. In other words, use of the wearable device 2 shown in FIG. 11 provides the following effects.
Visual information specific to dermatology can be obtained in real time.
Detailed operations and lighting manipulation can be performed.
Zoom-in and zoom-out can be performed.

Examples of biometric information acquired by the wearable device 2 shown in FIG. 11 include information about the visual image (e.g., skin surface image). In the example shown in FIG. 11, the medical facility terminal 4 displays a visual image of the skin indicated by the reference numeral D51. The type of image indicated by the reference numeral D41 can be changed when the doctor selects the button indicated by the reference numeral B51. When the visual image is displayed, a button may be displayed to control the on/off of the light (lighting) in the wearable device 2. In addition, the ranges or positions included in the image indicated by the reference numeral D41 can be changed or zoomed-in/out when the doctor presses (or touches) the button indicated by the reference numerals B52 to B54.

### <<Other Functions>>

In addition to the functions shown in FIGS. 6 to 11, biometric information acquired by the following devices (with a communication function), for example, can be obtained in real time.
Fetal heart rate, fetal movement, and uterine contraction pressure in online obstetric examinations (whether an NST measurement device with a ultrasound transducer is worn as the wearable device 2.)
Biometric information (e.g., weight, body fat, basal metabolism, muscle mass, BMI, and blood glucose level) acquired by the combination of a body composition analyzer and a wristwatch-type device
Biometric information (e.g., systolic blood pressure, diastolic blood pressure, and heart rate) acquired by a wristwatch-type device
Biometric information (e.g., oxygen saturation and heart rate) acquired by a pulse oximeter
Biometric information (e.g., casual blood glucose and continuous blood glucose) obtained by a blood glucose measurement device
Biometric information (e.g., electroencephalogram) acquired by an electroencephalogram measurement device
Biometric information acquired by other predetermined contactless devices

### <Advantageous Effects of This Embodiment>

According to the embodiments described above, doctors can acquire biometric information of a patient in real time during online medical examinations. It can also be compared with the patient's past biometric information. The use of a wearable device can reduce the burden on the patient (user) and also allows for acquisition of biometric information for a long period of time without interfering with the measurement operation. In addition, by obtaining patients' biometric information in real time during online medical examinations, patients can receive a diagnosis from a specialist regardless of where they live. This will enable flexible implementation of early detection of symptoms and prevention of recurrence, thereby reducing medical costs and improving the quality of life of people.

According to the embodiment described above, the doctor can remotely control the wearable device 2 to acquire biometric information as he/she intends, like in face-to-face medical examinations.

According to the embodiment described above, the protocol, API, and SDK for information that can be installed in any device (wearable device 2; hardware communication device) can be used to acquire biometric information of the patient, independently of the type of device. The information stored in the storage area of the device itself may be read by a reading terminal connected to the computer in order to enable indirect remote acquisition of medical information. For example, a blood pressure monitor may contain a recording medium such as FeliCa (registered trademark) so that the patient's condition may be obtained by reading the recording medium. In addition, a step instructed by the doctor via remote control may be pre-saved on a recording medium, so that the contents of the recording medium may be read by a predetermined hardware to allow the doctor to read the patient's biometric information in the step pre-specified by the doctor. The recording medium may be contactless or contact-type.

In addition, according to the embodiment described above, presenting abnormalities in biometric information detected by the alert unit (e.g., AI) to the doctor prevents them to be undetected and enables accurate diagnosis.

Further, according to the embodiment described above, the acquired biometric information can be stored in the storage unit to be accumulated as an electronic medical record. The stored data can be checked by the patients themselves using a patient terminal, for example. The accumulated data can also be used as various types of research data.

Further, according to the embodiment described above, the wearable device 2 can be rented or leased for online medical examinations so that widespread and general use of accurate online medical examinations can be achieved.

Although one embodiment of the present invention has been described above, the present invention should not be limited to the embodiment described above, and variations, modifications, and the like to the extent at which the purpose of the invention can be achieved are included in the present invention.

### Modification

In the embodiment described above, the alert unit detects abnormalities in biometric information and presents them to the medical facility terminal; however, the alert unit (AI) may learn patterns of tests in online medical examinations and alert the next time, or later, that any missing test is found. In addition, the patient may acquire biometric information every day at home, and based on the biometric information, the alert unit may present suggestions for improvements in their lifestyle habits.

In the embodiment described above, the size of the wearable device was not particularly mentioned; however, the wearable device may be sized to fit the patient's body shape, for example, S, M, and L. The different sensors may be made detachable so that they can be attached to existing clothing (e.g., pasted).

The aforementioned embodiment has taken as an example the case of an online medical examination between a doctor at a medical facility and a patient; however, this is not necessarily the case and periodic checkups at a nursing care facility (e.g., ophthalmological examinations), for example, can also be an application. In this case, it is preferable that a caregiver or nurse be on site to assist in the operation of the wearable device 2.

The aforementioned embodiment has described the example of transmitting biometric information and the like when instructions are received from the user, doctor, or the like; however, even without reception of instructions, wearing the wearable device and turning it on may allow the acquired biometric information to be automatically saved in the storage area set in the wearable device, and the information saved in the storage area of the wearable device to be then transmitted to the server at the time of its connection to the patient terminal via the network through the patient terminal (e.g., cell phone). This is a countermeasure to, for example, wearable device terminals that require 24-hour monitoring due to the disconnection of the communication environment.

In the aforementioned embodiment, the wearable device may also include a non-stress test (NST) measurement device with an ultrasound transducer used in online obstetric examinations.

In the aforementioned embodiment, the wearable device may include at least one or more selected from a measurement device that measures biometric information including body shape information when it is worn, and a measurement device that measures facial coloration. Here, the measurement device that measures biometric information including body shape information when worn is, for example, a full-body suit-type device. Examples of the measurement device that measures facial coloration include devices that measure facial coloration based on image information captured when the user wears glasses which have multiple colors represented on the frames.

In the aforementioned embodiment, the control information reception unit 35 (reception unit) may also accept diagnosis information regarding a specific diagnosis (e.g., asthma). In this case, the display control unit 34 (display control unit) may display, in an emphasized manner on the display (display unit) of the medical facility terminal 4 (second terminal), the information corresponding to the diagnosis information selected from among at least one or more selected from biometric information, drug information, and medical records. For example, if “asthma” information is received from a doctor as diagnosis information, information such as “breathing sounds”, which is biometric information corresponding to “asthma”, and “steroids”, which is drug information, may be displayed on the medical facility terminal 4.

Further, in the aforementioned embodiment, in a case of an initial online medical examination, the display control unit 34 (display control unit) may display information corresponding to the aforementioned diagnosis information on the display (display unit) of the medical facility terminal 4 (second terminal). To be specific, it is recommended that the information be grouped by medical department, for example, those related to blood pressure be picked up and displayed as information corresponding to the diagnosis information.

### Others

For example, the series of processes described above can be executed by hardware or by software. In other words, the functional configuration described above is merely illustrative and is not necessarily the case. In other words, it is sufficient if the information processing system has a function that enables the aforementioned series of processes to be executed as a whole, and what functional blocks are used to achieve this function is not necessarily like in the aforementioned examples. The locations of the functional blocks are not necessarily like in FIG. 4 and can be arbitrary. For example, the functional blocks of the server may be transferred to other terminals, devices, and the like. Conversely, the functional blocks of other terminals or devices may be transferred to the server or the like. One functional block may consist of hardware alone, software alone, or a combination of the two.

When a series of processes is executed by software, the programs constituting the software are installed on a computer or other device, from a network or recording medium. The computer may be a computer integrated in dedicated hardware. The computer may also be a computer capable of performing different functions by installing different programs, for example, a server as well as a general-purpose smartphone or personal computer.

The recording medium containing such a program consists not only of a removable medium (not shown in the drawing) that is distributed separately from the main unit of the device in order to provide the program to users and the like, but also of a recording medium or the like pre-installed in the main unit of the device to be provided to users and the like. Since the program can be distributed via a network, the recording medium may be mounted on or accessible to a computer connected or connectable to the network.

In this specification, the steps of describing a program recorded on a recording medium include not only processing that is performed chronologically according to their sequence, but also processing that is performed in parallel or individually without necessarily being processed chronologically. In this specification, the term “system” refers to an overall device consisting of multiple devices, multiple unit, and the like.

In other words, the information processing apparatus to which the present invention is applied can vary in different embodiments having the following configurations. In other words, provided is (1) an information processing apparatus including: an acquisition unit (e.g., acquisition unit 31) that acquires biometric information of a subject acquired by a sensor (including image-capturing sensor) provided in a first terminal attached to the subject and terminal information including a type of the first terminal; a storage unit (e.g., storage unit 18) that stores the biometric information and the terminal information that have been acquired; a display control unit (e.g., display control unit 34) that displays the biometric information and the terminal information on a display unit of a second terminal that is different from the first terminal; a reception unit (e.g., control information reception unit 35) that receives from the second terminal control information for the first terminal; and an instruction unit (e.g., control information transmission unit) that instructs the first terminal to control the first terminal based on the control information that has been received. Accordingly, this allows medical personnel and the like to accurately obtain biometric information of patients and the like in online medical examinations and the like.

(2) Regarding the information processing apparatus, the display control unit may display, on the display unit, an image showing the first terminal, an image showing a location of the sensor of the first terminal, and the biometric information acquired by the sensor, based on the biometric information and the terminal information. This allows, for example, doctors and the like to know exactly at which location (site) biometric information was acquired by which wearable device.

(3) The sensor may include a plurality of sensors, the first terminal may include the plurality of sensors, and the reception unit may receive from the second terminal the designation of one or more sensors selected from the plurality of sensors, and the instruction unit may instruct the first terminal to acquire biometric information corresponding to the designated sensor. This allows doctors and the like to acquire biometric information as they intend like in face-to-face medical examinations, by instructing it to acquire biometric information corresponding to the sensor at the desired location.

(4) The terminal information may include information about a location of the sensor in the first terminal, and a correspondence unit (e.g., correspondence unit 32) that establishes correspondences between the biometric information and the location of the sensor that has acquired the biometric information in the first terminal may be further provided. This allows doctors and the like to determine which sensor in the wearable device is acquiring the corresponding biometric information by checking the medical facility terminal.

(5) A first alert unit (e.g., alert unit 33) that presents an alert to the second terminal when the biometric information is outside a predetermined range may be further provided. This allows the patient to receive an appropriate medical examination in case of an abnormality in the biometric information without overlooking the abnormality.

(6) A second alert unit that presents an alert to the second terminal based on past biometric information regarding the subject may be further provided. This allows the doctor to compare past patient information and the patient to receive an appropriate medical examination in case of an abnormality in the biometric information without overlooking the abnormality.

(7) The first terminal may include a non-stress test (NST) measurement device with an ultrasound transducer used in online obstetric examinations. This allows for the transmission, storage, and viewing of non-stress test information in online obstetric examinations.

(8) The first terminal may include at least one or more selected from a measurement device that measures the biometric information including body shape information when worn, and a measurement device that measures facial coloration. This enables the acquisition of information such as body shape information and facial color, which would not be able to be acquired with conventional biometric information acquisition devices.

(9) The reception unit may receive diagnosis information regarding a specific diagnosis, and the display control unit may display, in an emphasized manner on the display unit of the second terminal, information corresponding to the diagnosis information selected from among at least one or more selected from the biometric information, drug information, and medical records. This allows for the automatic display of diagnosis information regarding the diagnosis.

(10) In a case of an initial online medical examination, the display control unit may display information corresponding to the diagnosis information on the display unit of the second terminal. This allows patient information to be displayed to doctors and the like in advance, like in a medical questionnaire.

(11) An aspect may be regarded as a method for controlling an information processing apparatus, including: an acquisition step of acquiring biometric information of a subject acquired by a sensor (including image-capturing sensor) provided in a first terminal attached to the subject, and terminal information including the type of the first terminal, a display control step of displaying the biometric information and the terminal information on a display unit of a second terminal that is different from the first terminal, a reception unit that receives from the second terminal control information for the first terminal, and an instruction step of instructing the first terminal to control the first terminal based on the control information that has been received.

(12) An aspect may be regarded as a computer program for causing a computer to execute: an acquisition step of acquiring biometric information of a subject acquired by a sensor (including image-capturing sensor) installed in a first terminal attached to the subject, and terminal information including the type of the first terminal,
a display control step of displaying the biometric information and the terminal information on a display unit of a second terminal that is different from the first terminal,
a reception unit that receives from the second terminal control information for the first terminal, and
an instruction step of instructing the first terminal to control the first terminal based on the control information that has been received.

### EXPLANATION OF REFERENCE NUMERALS

1: Server
2: Wearable device
3: Patient terminal
4: Medical facility terminal
11: CPU 18: Storage unit
19: Communication unit
31: Acquisition unit
32: Correspondence unit
33: Alert unit
34: Display control unit
35: Control information reception unit
36: Control information transmission unit

1. An information processing apparatus comprising:
an acquisition unit that acquires biometric information of a subject acquired by a sensor provided in a first terminal attached to the subject and terminal information including a type of the first terminal;
a storage unit that stores the biometric information and the terminal information that have been acquired;
a display control unit that displays the biometric information and the terminal information on a display unit of a second terminal that is different from the first terminal;
a reception unit that receives from the second terminal control information for the first terminal; and
an instruction unit that instructs the first terminal to control the first terminal based on the control information that has been received.

2. The information processing apparatus according to claim 1, wherein the display control unit displays, on the display unit, an image showing the first terminal, an image showing a location of the sensor of the first terminal, and the biometric information acquired by the sensor, based on the biometric information and the terminal information.

3. The information processing apparatus according to claim 1, wherein
the sensor comprises a plurality of sensors and the first terminal comprises the plurality of sensors,
the reception unit receives from the second terminal designation of one or more sensors selected from the plurality of sensors, and
the instruction unit instructs the first terminal to acquire biometric information corresponding to the designated sensor.

4. The information processing apparatus according to claims 1, wherein
the terminal information includes information about a location of the sensor in the first terminal, the information processing apparatus further comprising
a correspondence unit that establishes correspondences between the biometric information and the location of the sensor that has acquired the biometric information in the first terminal.

5. The information processing apparatus according to claim 1, further comprising a first alert unit that presents an alert to the second terminal when the biometric information is outside a predetermined range.

6. The information processing apparatus according to claim 1, further comprising a second alert unit that presents an alert to the second terminal based on past biometric information regarding the subject.

7. The information processing apparatus according to claim 1, wherein the first terminal includes a non-stress test (NST) measurement device with an ultrasound transducer used in online obstetric examinations.

8. The information processing apparatus according to claim 1, wherein the first terminal includes at least one or more selected from a measurement device that measures the biometric information including body shape information when worn, and a measurement device that measures facial coloration.

9. The information processing apparatus according to claim 1, wherein
the reception unit receives diagnosis information regarding a specific diagnosis, and
the display control unit displays, in an emphasized manner on the display unit of the second terminal, information corresponding to the diagnosis information selected from among at least one or more selected from the biometric information, drug information, and medical records.

10. The information processing apparatus according to claim 9, wherein in a case of an initial online medical examination, the display control unit displays information corresponding to the diagnosis information on the display unit of the second terminal.

11. A method for controlling an information processing apparatus, comprising:
an acquisition step of acquiring biometric information of a subject acquired by a sensor provided in a first terminal attached to the subject, and terminal information including the type of the first terminal;
a display control step of displaying the biometric information and the terminal information on a display unit of a second terminal that is different from the first terminal;
a reception unit that receives from the second terminal control information for the first terminal; and
an instruction step of instructing the first terminal to control the first terminal based on the control information that has been received.

12. A non-transitory computer readable medium storing a computer program for causing a computer to execute:
an acquisition step of acquiring biometric information of a subject acquired by a sensor provided in a first terminal attached to the subject, and terminal information including the type of the first terminal;
a display control step of displaying the biometric information and the terminal information on a display unit of a second terminal that is different from the first terminal;
a reception unit that receives from the second terminal control information for the first terminal; and
an instruction step of instructing the first terminal to control the first terminal based on the control information that has been received.
